(19) United States
(12) Patent Application Publication
(10) Pub. No.: US 2016/0256228 A1
(43) Pub. Date: Sep. 8, 2016

(54) INTERVENTIONAL SYSTEM

(71) Applicant: KONINKLIJKE PHILIPS N.V., Eindhoven (NL)

(72) Inventors: Jacob Roger Haartsen, Eindhoven (NL); Gert Wim 'T Hooft, Eindhoven (NL); Maurice Hubertus Elisabeth Van Der Beek, Eindhoven (NL); Franciscus Johannes Gerardus Hakkens, Eersel (NL)

(21) Appl. No.: 15/027,739

(22) PCT Filed: Sep. 30, 2014
(86) PCT No.: PCT/EP2014/070842

§ 371 Date: Apr. 7, 2016

(30) Foreign Application Priority Data
Oct. 16, 2013 (EP) 13188867.9

(51) Int. Cl. A61B 34/30; A61M 25/01; A61B 34/20

(52) U.S. Cl. CPC A61B 34/30; A61B 34/20; A61M 25/0141; A61B 2034/301; A61B 2034/2055

(57) ABSTRACT

The invention relates to an interventional system (1) for performing an interventional procedure. An interventional instrument (5) like a catheter comprises a bendable portion (12), which is bendable by a bending element (11), and an OSS fiber (10) for generating OSS signals being indicative of the degree of bending of the bendable portion. The actual degree of bending of the bendable portion is determined based on the generated OSS signals and the bending element is controlled depending on the actual determined degree of bending. By using OSS, the actual real degree of bending of the bendable portion of the interventional instrument can very accurately be determined. Moreover, since the bending element is controlled based on this very accurately determined degree of bending, the control of the bending element and, thus, of the interventional instrument can be very accurately.

### FIELD OF THE INVENTION

The invention relates to an interventional system, method and computer program for performing an interventional procedure.

### BACKGROUND OF THE INVENTION

WO 2011/143338 A1 a robotic system comprising a first instrument, a base that the first instrument is coupled to so that the first instrument moves when the base moves, a base controller means for causing the base to be moved so as to optimize a work space of the first instrument as the first instrument moves and a first instrument controller for moving the first instrument according to a commanded movement while compensating for movement of the base.

WO 2009/023801 A1 discloses a robotic medical instrument system comprising a controller configured to control the actuation of at least one servo motor, an elongate instrument configured to move in response to the actuation of the at least one servo motor and an optical fiber having a distal portion coupled to a distal portion of the instrument, wherein the distal portion of the optical fiber comprises a fiber core having a plurality of axially-spaced Bragg gratings. The robotic medical instrument further comprises a detector operatively coupled to a proximal end of the optical fiber and configured to detect respective light signals reflected by the axially-spaced Bragg gratings, wherein the controller controls the movement of the instrument based at least in part upon a geometric configuration of the distal portion of the instrument which is determined based on an analysis of the detected light signals.

U.S. Pat. No. 8,347,738 B2 discloses an active interventional catheter comprising a force and position sensor. The catheter comprises a first end to be inserted into a body lumen and a second end to be outside of the body lumen, wherein the sensor is incorporated proximal to the first end of the catheter. The electrical resistance across the sensor changes in accordance with a displacement of the first end of the catheter, thereby providing a measure for the force on and the position of the first end of the catheter. This measure can be used to determine force information to be transmitted to a physician and to determine the position of the first end of the catheter, which may be used as a position feedback during a minimally invasive procedure, in order to allow for a closed-loop control of the position of the first end of the catheter under computer-aided guidance. However, controlling the position of the first end of the catheter based on the changes of the electrical resistance across the sensor is not very accurate.

### SUMMARY OF THE INVENTION

It is an object of the present invention to provide an interventional system, method and computer program for performing an interventional procedure, which allows for an improved control of an interventional instrument of the interventional system. In a first aspect of the present invention an interventional system for performing an interventional procedure is presented, wherein the interventional system comprises:
a handheld interventional instrument comprising a bendable portion, wherein the interventional instrument is equipped with a bending element being adapted to bend the bendable portion and with an optical shape sensing (OSS) fiber for generating OSS signals being indicative of the degree of bending of the bendable portion, wherein the bending element comprises a smart material connected to the bendable portion, wherein the smart material is adapted to change its spatial configuration depending on an external stimulus, in order to change the degree of bending of the bendable portion,
a bending determination unit for determining the degree of bending of the bendable portion based on the generated OSS signals,
a desired bending providing unit for providing a desired degree of bending,
a control unit for controlling the bending element in a control loop depending on the determined degree of bending and on the desired degree of bending such that the determined degree of bending is similar to the desired degree of bending by providing an external stimulus to the smart material.

Since the interventional instrument is equipped with an OSS fiber for generating OSS signals being indicative of the degree of bending of the bendable portion, wherein the degree of bending of the bendable portion is determined based on the generated OSS signals, the actual real degree of bending of the bendable portion of the interventional instrument can very accurately be determined. Moreover, since the bending element is controlled based on this very accurately determined degree of bending, the control of the bending element and, thus, of the interventional instrument can be very accurate.

The interventional instrument is preferentially a catheter or a guidewire. The interventional instrument can be equipped with one or several bending elements, wherein the control unit can be adapted to control the one or several bending elements depending on one or several determined degrees of bending. In particular, each bending element may be individually controlled based on a corresponding individually determined degree of bending.

In an embodiment the interventional instrument comprises several bendable portions and several bending elements for bending the several bendable portions, wherein the bending determination unit is adapted to determine the degrees of bending of the several bendable portions and wherein the control unit is adapted to control the respective bending element depending on the respective determined degree of bending. Thus, the degrees of bending of several bendable portions can be individually accurately controlled, in order to allow for many different accurate positions and shapes of the interventional instrument.

Moreover, in an embodiment the interventional instrument comprises several bending elements for bending the same bendable portion of the interventional instrument, wherein different bending elements are adapted to bend the same bendable portion in different directions and wherein the control unit is adapted to control the bending elements depending on the determined degree of bending. Thus, the same bendable portion can be bent accurately and in different directions by individually controlling the respective bending element. Also this leads to different accurate positions and shapes of the interventional instrument. In an embodiment the interventional instrument comprises several bendable portions, wherein each bendable portion comprises several bending elements for bending the respective bendable portion in different directions.

The bending element comprises a smart material connected to the bendable portion, wherein the smart material is adapted to change its spatial configuration depending on an external stimulus, in order to change the degree of bending of the bendable portion, wherein the control unit is adapted to control the bending element depending on the determined degree of bending by providing the external stimulus depending on the determined degree of bending. The spatial configuration of the smart material, which is changed depending on the external stimulus, is, for instance, the shape and/or the volume and/or the length, et cetera of the smart material. The external stimulus may be the temperature, an electric field, a magnetic field, et cetera. Preferentially, the smart material is a shape memory alloy (SMA) wire, wherein the shape of the SMA wire changes in response to heat, which may be provided by the control unit as the external stimulus, wherein the heat may be provided by providing an electrical current, in order to resistively heat the SMA wire. The bending element can also comprise another smart material like an electroactive polymer (EAP). The smart material may be embedded in the interventional instrument, in particular, if the interventional instrument is a catheter, the smart material may be embedded in a wall of the catheter. If the bending element is based on the smart material, the bending element can be relatively small and the bendable portion can be bent relatively fast.

The interventional system comprises a desired bending providing unit for providing a desired degree of bending, wherein the control unit is adapted to control the bending element in a control loop depending on the determined degree of bending and on the desired degree of bending such that the determined degree of bending is similar to the desired degree of bending. The control loop compares the determined actual degree of bending and the desired degree of bending and corrects the actual degree of bending, if the determined actual degree of bending and the desired degree of bending are not similar. If the interventional instrument is equipped with several bending elements, for each bending element a desired degree of bending may be provided and each bending element may be individually controlled in an individual control loop depending on the determined respective actual degree of bending and on the respective desired degree of bending such that the respective determined actual degree of bending is similar to the respective desired degree of bending. This allows for a further improved control of the interventional instrument.

In a preferred embodiment the desired bending providing unit is adapted to provide a user interface for allowing a user to input the desired degree of bending and to provide the input desired degree of bending. The user interface may be integrated in a handle of the interventional instrument. For instance, the desired bending providing unit can be adapted to provide a dial mechanism in a catheter handle as a user interface, in order to allow the user to input the desired degree of bending. In this way the interventional instrument can very accurately be controlled by the user.

In an embodiment the interventional system further comprises a desired position providing unit for providing a desired position of the interventional instrument, wherein the desired bending providing unit is adapted to determine the desired degree of bending based on the provided desired position of the interventional instrument. In particular, the interventional system further comprises a position determination unit for determining the position of the interventional instrument, wherein the desired bending providing unit is adapted to determine the desired degree of bending further based on the determined position of the interventional instrument. The desired position of the interventional instrument may be a position of the entire interventional instrument or a position of a part of the interventional instrument like a position of a tip of a catheter.

The desired position providing unit may comprise a user interface for allowing a user to input the desired position of the interventional instrument and to provide the input desired position of the interventional instrument. For instance, the user interface can show an inner structure of a living being like a person or an animal and can allow the user to indicate the desired position on the shown inner structure, or the user interface can be adapted to allow the user to indicate that the actual position should be provided as the desired position.

The position determination unit can be adapted to determine an absolute position of the interventional instrument by using OSS or by using another technique, or to determine a relative position of the interventional instrument like a position of the interventional instrument relative to an inner wall of a living being. The position determination unit can be adapted to use sensor signals received from position sensors for determining the position of the interventional instrument. The position sensors may be ultrasound sensors, force sensors, et cetera. The desired bending providing unit can be adapted to determine the desired degree of bending such that a possible distance between the provided desired position of the interventional instrument and the determined position of the interventional instrument is reduced. This reduction of the distance between the provided desired position and the determined position can be performed in a control loop, until the determined position and the provided desired position are equal. The control loop may comprise providing the desired position of the interventional instrument, determining the actual position of the interventional instrument, determining a desired degree of bending required for reducing a possible distance between the provided desired position and the actual determined position, determining the actual degree of bending of the bending element and controlling the bending element depending on the actual determined degree of bending and on the desired degree of bending such that the determined degree of bending is equal to the desired degree of bending. This can allow for a further improved control of the interventional instrument. After the interventional instrument has reached the provided desired position, the control procedure can continue, in order to ensure that the interventional instrument remains at the provided desired position, thereby correcting for, for instance, movements or tremor of a physician, or in order to move the interventional instrument to a further provided desired position.

The bending determination unit is preferentially adapted to determine the curvature and/or the radius of curvature and/or the bending angle of the bendable portion as the degree of bending. The curvature, the radius of the curvature, and the bending angle of the bendable portion are very good measures for the degree of bending of the bendable portion, which therefore lead to a further improved control of the bending element. These measures for the degree of bending are particularly suitable for controlling the bending element in a control loop depending on the respective determined actual degree of bending and on the respective desired degree of bending such that the respective determined actual degree of bending is similar to the respective desired degree of bending.

In another aspect an interventional instrument for an interventional system as defined in claim 1 is presented, wherein the interventional instrument comprises a bendable portion and is equipped with a bending element being adapted to bend the bendable portion and with an OSS fiber for generating OSS signals being indicative of the degree of bending of the bendable portion, wherein the bending element comprises a smart material connected to the bendable portion, wherein the smart material is adapted to change its spatial configuration depending on an external stimulus, in order to change the degree of bending of the bendable portion.

In a further aspect a control unit for being used by an interventional system as defined in claim 1 is presented, wherein the control unit is adapted to control the bending element of the interventional instrument of the interventional system depending on the degree of bending determined by the bending determination unit of the interventional system and on the desired degree of bending such that the determined degree of bending is similar to the desired degree of bending by providing an external stimulus to the smart material.

In another aspect of the present invention an interventional method for performing an interventional procedure is presented, wherein the interventional method comprises:
generating OSS signals being indicative of a degree of bending of a bendable portion of a handheld interventional instrument which is equipped with an OSS fiber for generating the OSS signals and with a bending element for bending the bendable portion, wherein the bending element comprises a smart material connected to the bendable portion, wherein the smart material is adapted to change its spatial configuration depending on an external stimulus, in order to change the degree of bending of the bendable portion,
determining the degree of bending of the bendable portion based on the generated OSS signals by a bending determination unit,
providing a desired degree of bending by a desired bending providing unit, and
controlling the bending element in a control loop depending on the determined degree of bending and on the desired degree of bending such that the determined degree of bending is similar to the desired degree of bending by a control unit providing an external stimulus to the smart material.

In a further aspect of the present invention a computer program for performing an interventional procedure is presented, wherein the computer program comprises program code means for causing an interventional system as defined in claim 1 to carry out the steps of the interventional method as defined in claim 11, when the computer program is run on a computer controlling the interventional system.

It shall be understood that the interventional system of claim 1, the interventional instrument, the control unit, the interventional method of claim 11 and the computer program of claim 12 have similar and/or identical preferred embodiments, in particular, as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of an interventional system for performing an interventional procedure,

FIGS. 2 to 4 schematically and exemplarily illustrate a bendable portion of a catheter of the interventional system,

FIG. 5 schematically and exemplarily illustrates a control of several bendable portions of the catheter,

FIG. 6 shows a flowchart exemplarily illustrating an embodiment of an interventional method for performing an interventional procedure, and

FIG. 7 shows schematically and exemplarily an embodiment of a tip of a catheter.

### DETAILED DESCRIPTION OF EMBODIMENTS

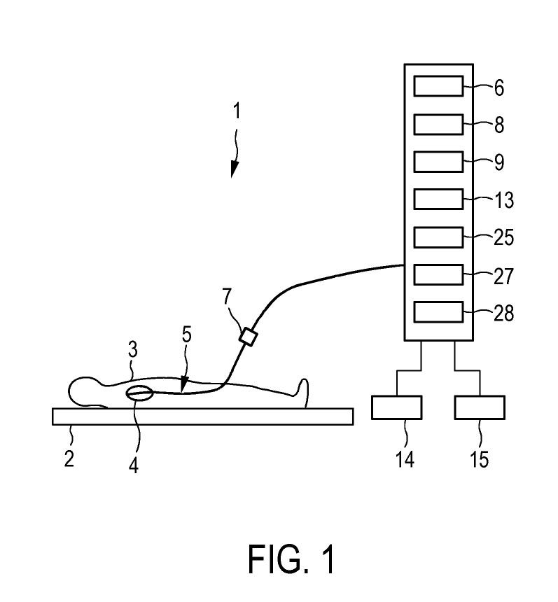

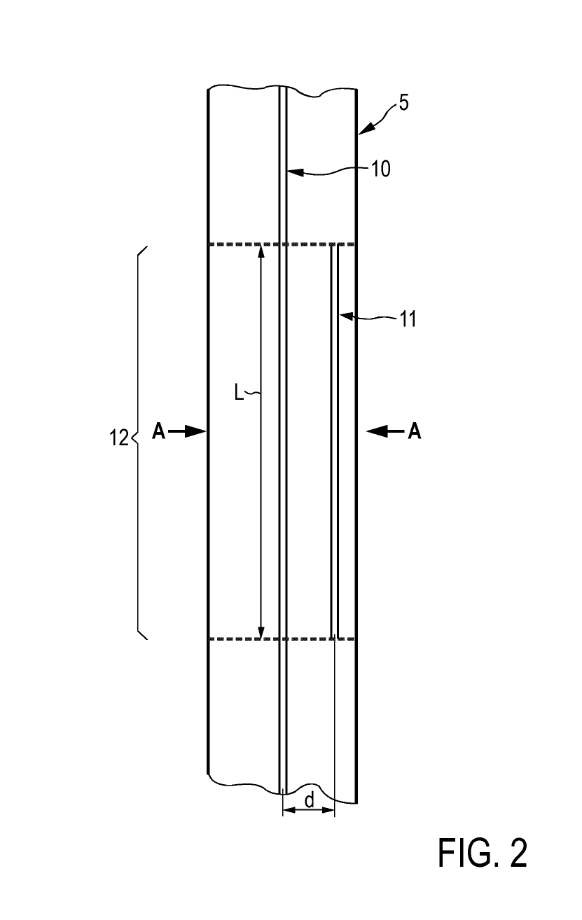

FIG. 1 shows schematically and exemplarily an interventional system for performing an interventional procedure. In this embodiment the interventional system 1 is adapted to perform an interventional procedure within a heart 4 of a person 3 lying on a support means like a patient table 2. The interventional system 1 comprises a handheld catheter 5 comprising several bendable portions 12, wherein one of these bendable portions 12 is schematically and exemplarily illustrated in FIG. 2.

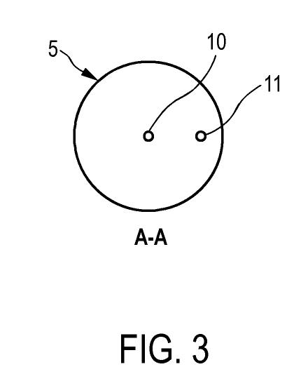

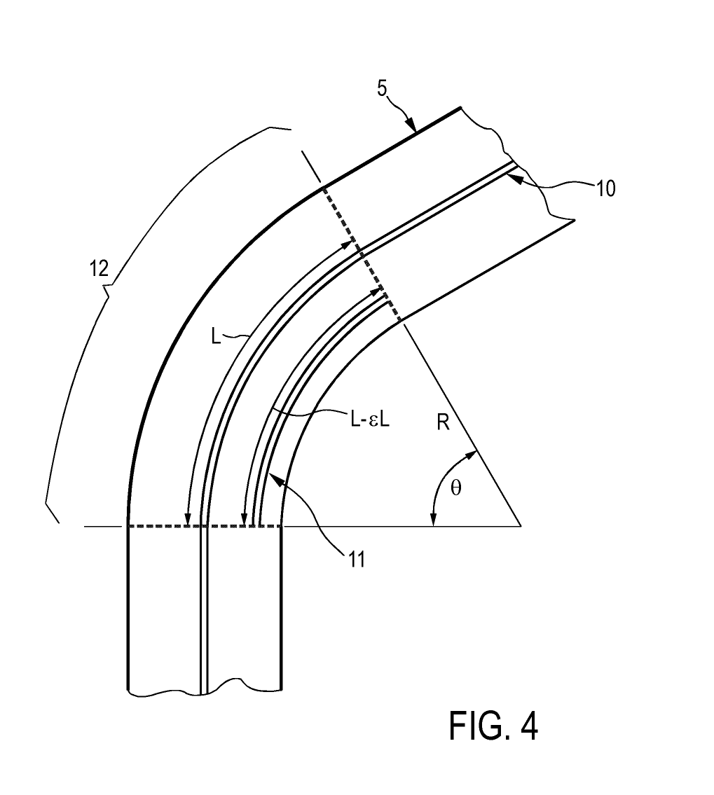

Each bendable portion 12 comprises an SMA wire 11 forming a bending element. Moreover, the catheter 5 is equipped with an OSS fiber 10 for generation OSS signals being indicative of the degree of bending of the respective bendable portion 12. In this embodiment the OSS fiber 10 is centrally located within the catheter 5 and the SMA wire 11 is arranged at an offset position relative to the center of the catheter 5 as schematically and exemplarily illustrated in FIG. 3, which shows a cross sectional view of the catheter 5 illustrated in FIG. 2 at the location indicated by the reference letter A. It should be noted that the catheter 5 comprises more components than the components shown in FIGS. 2 and 3 like components for sensing heart characteristics and/or for treating the heart, which may be arranged within one or more several lumina of the catheter 5. Furthermore, the catheter 5 comprises wires electrically connected to the SMA wires 11, in order to allow a control unit 8 to control the SMA wires 11. These further components are not shown in FIGS. 2 and 3 and also not in FIG. 4, which just illustrate the bending element used for bending the bendable portion and the OSS fiber used for determining the degree of bending of the bendable portion, for clarity reasons.

The interventional system 1 further comprises a bending determination unit 6 for determining the degree of bending of the respective bendable portion 12 based on the generated OSS signals, wherein the control unit 8 is adapted to control the respective SMA wire 11 depending on the respective determined degree of bending. In particular, the control unit 8 is adapted to apply a voltage or current to the respective SMA wire, in order to heat the respective SMA wire, wherein the heat is caused by the electrical resistance of the respective SMA wire. Thus, in this embodiment heat is used as an external stimulus for changing the shape of the respective SMA wire.

The interventional system 1 further comprises a desired bending providing unit 7 for providing desired degrees of bending of the bendable portions 12 of the catheter 5, wherein the control unit 8 is adapted to control the respective SMA wire 11 of a respective bendable portion 12 in a control loop depending on the respective determined degree of bending and on the respective desired degree of bending such that the respective determined degree of bending is similar to the respective desired degree of bending. The desired bending providing unit 7 may be adapted to provide a user interface for allowing a user to input the desired degrees of bending and to provide the input desired degrees of bending. For instance, the desired bending providing unit 7 can be adapted to provide a dial mechanism in a handle of the catheter as the user interface, in order to allow the user to input the desired degrees of bending. However, in another embodiment the desired bending providing unit can also be adapted to provide a graphical user interface allowing the user to input desired degrees of bending by using an input unit 14 and a display 15. The input unit 14 may include a keyboard, a computer mouse, a touch screen, et cetera.

The bending determination unit 6 is preferentially adapted to determine the curvature and/or the radius of curvature and/or the bending angle of the respective bendable portion 12 as a respective degree of bending. FIG. 4 schematically and exemplarily illustrates these preferred degrees of bending.

FIG. 4 shows the bendable portion 12, which is shown in FIG. 2, after the bendable portion has been bent. In FIG. 4 θ indicates the bending angle, R indicates the radius of curvature, wherein the curvature κ itself can be defined by the inverse radius of curvature R. Moreover, in FIG. 2 reference letter L indicates the length of the bendable portion 12, the length of the OSS fiber 10 within the bendable portion 12 and the length of the SMA wire 11. In FIG. 4 showing a situation, in which the bendable portion 12 has been bent, the part of the OSS fiber 10 within the bendable portion 12 still has the length L, whereas the bent SMA wire 11 has a reduced length of L−εL.

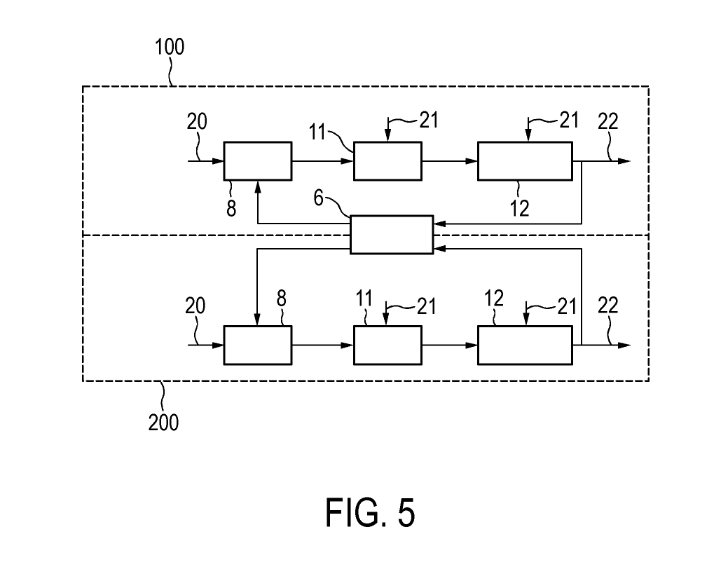

In the following the control of the bending elements, in particular, of the SMA wires 11, will exemplarily be described in more detail with reference to FIG. 5.

FIG. 5 illustrates the control of two bendable portions of the catheter 5, i.e. a first control 100 and a second control 200. The controller 8 receives the provided desired degree of bending, which is indicated by the arrow 20. The controller 8 also receives from the bending determination unit 6 the actual determined degree of bending of the respective bendable portion and compares the desired degree of bending and the determined actual degree of bending for generating a control signal for controlling the bending element 11. In this embodiment the control signal is a voltage applied to the SMA wire forming the bending element 11, wherein the applied voltage depends on the desired degree of bending and the determined actual degree of bending and is determined such that the respective bendable portion is bent in accordance with the provided desired degree of bending. The bending of the bending element 11 may also be influenced by disturbances indicated by the arrow 21. The bending of the bending element 11 leads to a force applied to the respective bendable portion 12 such that the bendable portion 12 is bent. Also this bending of the respective bendable portion 12 may be influenced by disturbances indicated by the arrow 21. OSS signals, which are indicative of the degree of bending of the respective bendable portion 12, are generated and provided to the bending determination 6 for allowing the bending determination unit 6 to determine the actual degree of bending of the respective bendable portion. The respective control loop may be performed, until a deviation between the respective desired degree of bending and the respective determined actual degree of bending is minimized. The result of the respective control loop is a respective final degree of bending of the respective bendable portion 12 indicated by the respective arrow 22.

If in an embodiment that interventional instrument comprises a single bendable portion with a single bending element only, only one of the controls 100, 200 described above with reference to FIG. 5 may be present. If more than two bendable portions with corresponding bending elements are present, the interventional system can comprise a corresponding number of the controls described above with reference to FIG. 5.

The interventional system 1 further comprises a treating unit 9 for treating the heart 4 of the person 3 at a location, to which the tip of the catheter 5 has been moved. For instance, in an embodiment the tip of the catheter 5 can comprise an electrode electrically connected to a radio frequency (RF) source of the treating unit 9 via a wire arranged within the catheter 5, in order to apply RF energy to the desired location within the heart 4, for instance, in order to apply a cardiac ablation procedure. However, in other embodiments the treating unit 9 can be adapted to perform another kind of treatment.

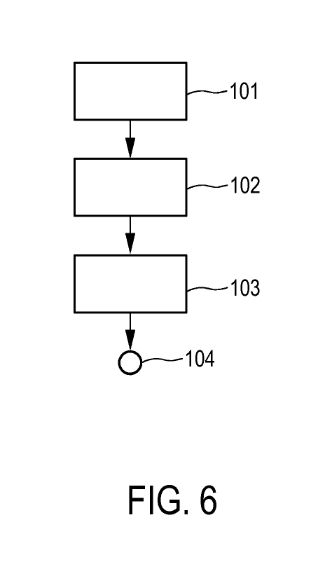

In the following an embodiment of an interventional method for performing an interventional procedure will exemplarily be described with reference to a flowchart shown in FIG. 6.

In step 101 the catheter is introduced into the person 3 and moved within the person 3 such that the tip of the catheter 5 reaches a desired location within the heart of the person 3. During the movement of the catheter 5 within the person OSS signals are generated being indicative of the degree of bending of the bendable portions of the catheter, the degrees of bending of the bendable portions are determined based on the generated OSS signals and the bending elements for bending the bendable portions are controlled depending on the determined degrees of bending, in order to allow for an accurate positioning of the tip of the catheter 5 at a desired location within the heart of the person. The generated OSS signals can be further used, for instance, by the bending determination unit 6 for determining the position of the catheter, especially of the tip of the catheter, within the person. This position of the catheter can be overlaid on a pre-interventional image of the person like a pre-interventional computed tomography or magnetic resonance image, in order to show the position of the catheter relative to the anatomy of the person. The pre-interventional image is registered with the OSS detection system, i.e. with the position of the catheter determined by using the OSS signals, by using known registration procedures. For example, during a pre-interventional registration procedure an imaging system used for generating the pre-interventional image of the person can also be used for generating a registration image showing the catheter, before the catheter is introduced into the person, wherein the position of the catheter in the reference image and the position of the catheter as determined by using the OSS signals can be used for the registration. The pre-interventional image can be provided by an image providing unit 13, which may be a storing unit, in which the pre-interventional image has been stored and from which the pre-interventional image can be retrieved for providing the same.

In step 102 the treating unit 9 is used for treating the heart at the desired location. For instance, RF energy can be applied to the desired location within the heart, for instance, in order to apply a cardiac ablation procedure. In step 103 the catheter is removed from the person, whereupon the method ends in step 104.

The interventional system 1 may further comprise a desired position providing unit 25 for providing a desired position of the catheter 5 and a second desired bending providing unit 28 that may be adapted to determine desired degrees of bending of the bendable portions based on the provided desired position of the catheter 5 and to provide the determined desired degrees of bending. The interventional system 1 may further comprise a position determination unit 27 for determining the position of the catheter 5, wherein the second desired bending providing unit 28 may be adapted to determine the desired degrees of bending further based on the determined position of the catheter 5.

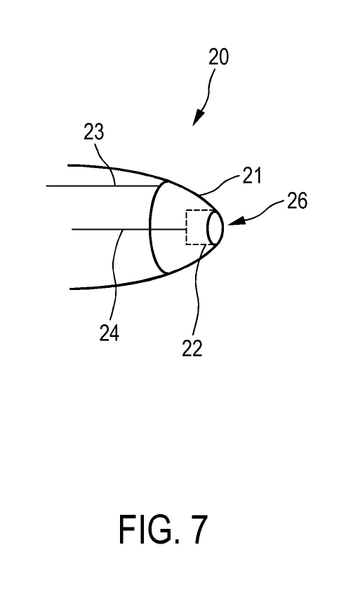

In an embodiment the tip 20 of the catheter 5 may comprise a position sensor 22 as schematically and exemplarily illustrated in FIG. 7. For instance, the tip 20 may comprise an ablation electrode 21 with a central opening 26, through which the position sensor 22, which might be an ultrasound sensor or a force sensor, can sense the position of an inner wall like a heart wall. Sensing signals generated by the position sensor 22 can be transmitted to the position determining unit 27 for determining the position of the tip 20 of the catheter 5 relative to the inner wall of the person 3 via an electrical connection 24 like a wire. An electrical connection 23 may be used for transmitting the RF energy to the ablation electrode 21.

The catheter 5 can be moved to a desired location within the heart 4 such that the tip 20 of the catheter 5 has a desired position relative to an inner wall of the heart 4. After the tip 20 of the catheter 5 has reached the desired position relative to the inner wall of the heart 4, the user may indicate this position as a desired position by using a user interface provided by the second desired position providing unit 25, wherein this indicated position can be stored in and provided by the second desired position providing unit 25. In order to keep the tip 20 of the catheter 5 at this desired position, a control loop can be applied, wherein the real position of the tip 20 of the catheter 5 is continuously determined by the position determination unit 27 based on sensor signals received from the position sensor 22, wherein the second desired bending providing unit 28 determines desired degrees of bending of the bendable portions of the catheter 5 based on the provided desired position and the determined real position of the tip 20 of the catheter 5 such that a possible deviation of the determined real position from the desired position is corrected, wherein the bending determination unit 6 determines the actual degrees of bending and wherein the control unit 8 controls the bending elements of the catheter 5 depending on the actual determined degrees of bending and on the desired degrees of bending such that the determined actual degrees of bending becomes equal to the desired degrees of bending. Such a control loop can be used, for instance, to keep the tip 20 of the catheter 5 in contact with an inner wall of the heart 4 or of a blood vessel. The control loop can also be used for other purposes. For instance, if the tip 20 of the catheter 5 comprises a force sensor, the control loop can be used to keep the tip of the catheter with a fixed force against the wall of, for instance, the heart or the blood vessel.

In a further embodiment the desired position providing unit 25 may be adapted to provide a user interface allowing the user to input a desired position of the catheter, in particular, of the tip of the catheter, within the person 3. For instance, the desired position providing unit 25 can be adapted to show a pre-interventional image of the heart 4 of the person 3 on the display 15, wherein the pre-interventional image is registered with the OSS detection system by using known registration procedures, and to allow the user to indicate one or several desired positions on the pre-interventional image. After the one or several desired positions have been indicated by the user, they can be stored in and provided by the desired position providing unit 25. The interventional system can then perform a control loop for moving the catheter to a desired position, wherein the bending determination unit 6, which in this case may also be regarded as being a position determination unit, determines the actual position of the catheter by OSS, wherein the second desired bending providing unit 28 determines desired degrees of bending of the bendable portions of the catheter 5 based on the determined actual position of the catheter and the desired position of the catheter, wherein the bending determination unit 6 determines the actual degrees of bending of the bendable portions of the catheter 5 and wherein the control unit controls the bending elements of the catheter depending on the determined actual degrees of bending and the desired degrees of bending such that the bending elements reach the desired degrees of bending. If a desired position has been reached, the control loop may be used again to move the catheter to a further position or to keep the catheter at the actual desired position. The control loop can therefore be used to keep the catheter at a fixed position by correcting, for instance, movements or tremor, or to perform an automated sequence of catheter movements, in particular, of tip movements, which may be performed during an ablation procedure.

Although in above described embodiments the interventional procedure is a cardiac ablation procedure, in other embodiments the procedure can also be another one. In particular, the interventional system and method can be adapted to perform another Minimally Invasive Vascular Surgery (MIVS) procedure, i.e. another surgery using minimally invasive instruments such as a catheter and a guidewire. For instance, they can be adapted to perform a Fenestrated EndoVascular aortic Aneurism Repair (FEVAR) procedure.

Catheter maneuverability has a strong influence on both the procedure time as well as the risk of complications such as perforation. However, conventional non-steerable catheters are often difficult to maneuver and control for the following reasons. The contact friction between the catheter and vessels may cause stick-slip phenomena, leading to hysteresis and sudden jumps in tip movement, with reduced control of the tip as a consequence. Hence, some locations are unreachable or are only reached after a lengthy procedure. Moreover, conventional catheters have a fixed stiffness and an uncontrollable distal shape. Therefore, in order to reach the desired location, surgeons often have to use trial-and-error to choose the catheter with the correct stiffness and distal shape. Again, such catheter replacements lead to extended operating times, and the repeated extraction and insertion can do serious harm to the patient and increase the risk of infection.

For these reasons, a steerable catheter is used. In an embodiment the steerable catheter may have the ability to vary its distal shape and distal bending stiffness. For example, the steerable catheter may comprise multiple segments, i.e. bendable portions, in the distal part of the catheter that can be individually bent by using bending elements for facilitating entering tortuous and complex vessels. The steerable catheter may use smart materials as described above, or it may be a magnetically and/or pull-wire steered catheter.

If the catheter is magnetically steered, it may have small magnets embedded in the tip as bending elements, and may be navigated by a magnetic field generated by guiding magnets beside the patient. The magnetically steered catheter enables very accurate steering and hence reduced risk of tissue trauma.

If the catheter is pull-wire steered, it may have one or more pull-wires running along the length of the catheter as bending elements. At the distal end the pull-wires may be fixed to the catheter tip offset from the neutral line to be able to apply a bending moment, whereas at the proximal end they may be mounted to an actuation mechanism in a catheter handle for manually controlling the pulling force. In this embodiment the catheter tip is designed such that it is much more flexible than the shaft such that the tip bends whereas the shaft remains nearly unbent upon pulling one of the pull-wires.

In an embodiment the catheter is adapted such that the tip or tip segments can be locally actuated, wherein (1) the actuation forces only act on the segment, i.e. on the bending portion, to be bent, thereby enabling the use of a shaft with a small bending stiffness, (2) stick-slip phenomena are fully or drastically reduced because either there is a much smaller contact area between actuator and lumen or there is no slip between actuator and lumen at all, (3) individual segments can independently be actuated, (4) in combination with an ASIC only two (power/signal, ground) or three electrical wires (signal, power, ground) run from the distal to the proximal part to address the individual actuators. Also in this embodiment preferentially smart materials are used for actuators, i.e. for bending elements, that are small, powerful and fast enough to locally actuate the tip, or segments, of the catheter. The smart materials may be SMAs or EAPs.

The smart material actuators may suffer from a non-linear behavior, i.e. the output of the actuator (strain) may not be directly proportional to its input (voltage, current, heating power et cetera). For example, the temperature-strain relation of an SMA wire may be strongly non-linear, may show quite some hysteresis and may be load dependent.

The catheter comprising the smart material actuators, i.e. the bending elements with the smart materials or formed by the smart materials, is therefore combined with an actuator control strategy that relies on an actual strain measurement that is input to a feedback control loop. The actual strain measurements are performed by using an OSS fiber as feedback. This has a number of advantages above the use of, for instance, conventional strain gauges or silicon strain sensors. Because of its form factor the fiber is relatively easy to integrate in a catheter, if not already present for shape reconstruction. Moreover, the bending of multiple segments can be measured with a single fiber. Furthermore, there is no need for electrical wires, interconnects and electrical contacts. Especially making reliable electrical contacts to sensors that will be subjected to strain can be a very challenging task at these small dimensions and difficult form factor. In addition, the sensor signal is integrated over the entire actuated segment giving a much more accurate measure for the curvature of the segment, instead of a local strain measurement. Also, shape control can be performed in parallel to shape reconstruction as the data for shape control and shape reconstruction are extracted from the same raw sensor signals, i.e. both rely on the underlying principle of measuring strain.

When the OSS fiber is integrated in the catheter, OSS technology enables the reconstruction of the three dimensional shape of the catheter. Basically, such a fiber comprises multiple cores of which one straight center core located on the neutral axis and three out-of-center helically wound cores are used for the shape reconstruction. Upon bending, the three out-of-center cores experience strain; a core on the inside of the bend is compressed, while a core on the outside of the bend is stretched. Since they are helically wound, the average strain measurement of the three out-of-center cores is a measure that can be used to account for twist. The center core serves as a reference to correct for mechanical and temperature induced axial strain. The strain of each core is measured using Optical Frequency Domain Reflectometry (OFDR). Here, depending on the type of fiber, interference patterns of reflected light from Fiber Bragg gratings or by Rayleigh scattered light from small intrinsic refractive index fluctuations in the fiber are analyzed. In the case of a fiber with gratings, multiple of these gratings are located in the cores along the length of the fiber. Light from a linearly swept tunable laser source that is coupled into the cores of the fiber is reflected at these gratings with a spectrum centered at the Bragg wavelength. At the interferometer this spectrum interferes with the spectrum from the reference path creating a fringe pattern of which the number of fringes as a function of time is proportional to the distance between the reference path and the grating (so interference from a grating that is located further away from the reference path gives a higher modulation frequency). The measured signal at the detector is a combination of all fringe frequencies of which the specific frequency belonging to each grating can be determined by a Fourier transformation, from which the grating locations can be determined. Since the signal from each grating is now isolated, the spectrum from each of the gratings can again be inverse Fourier transformed to analyze the individual spectra. As stretching of a grating shifts the Bragg wavelength, this is a measure for the local strain of the fiber. Now, taking into account axial strain and torsion, local bending angles can be calculated for each fiber segment (for a spectral scan of 20 nm at a central wavelength of 1540 nm the smallest fiber segment has a length of about 40 um). From this, an average bending angle and an average curvature of a larger segment along the length of the fiber can be calculated.

The interventional system is preferentially adapted to use an OSS fiber in combination with a smart material based actuator, i.e. bending element, to controllably bend one or multiple segments of a catheter. The bending of a segment is sensed with the OSS fiber that is integrated in the catheter. The deformation, which can be expressed as a curvature κ (or radius of curvature R=1/κ) of the segment, may be compared with a desired curvature (set point) that may be set by the surgeon, for instance, by manually adjusting a dial mechanism in the catheter handle. The error between the actual and the desired curvature can be input to the controlling unit, which can comprise a PID controller, but also, for example, a controller based on pulse-width modulation. The controlling unit may convert the error between the actual and desired curvature into a control signal, for instance, electrical power in the case of an SMA actuator, to control the actuator. Based on this signal the actuator, i.e. the bending element, may adapt its strain, which consequently may lead to a new curvature of the segment. Disturbances such as temperature or heat loss variations in the case of an SMA actuator and external forces may act on the actuator and the catheter segment.

FIGS. 2 to 4 illustrate in a simple practical case how the actuator strain, radius of curvature and bending angle may be related. Here, a segment of the catheter 5 is depicted with an OSS fiber 10 located in the neutral line of the catheter 5. An SMA wire 11 of length L is located at a distance d from the neutral line of the catheter 5. The relation between the wire strain ε=ΔL/L and the radius of curvature R can be approximated by

$$R \approx \vert \frac{d}{ɛ} \vert . \qquad ( 1 )$$

This is an approximation, because the bending moment is accompanied by a net axial force, thereby shifting the neutral line away from the SMA wire 11. However, for simplicity it is assumed that this effect to be small. Also, it is unlikely that the fiber is always located on the neutral line, however the associated error is small as long as d<<R. The angle θ [°] over which the segment of length L bends is given by

$$θ = \frac{180 L}{π R} \approx \vert \frac{180 ɛ L}{π d} \vert . \qquad ( 2 )$$

Equations (1) and (2) show that both the radius of curvature (or curvature κ=1/R) and the bending angle can be used as a control parameter, as they are inversely proportional and proportional to the strain of the actuator, respectively.

The catheter can comprise one or multiple bendable segments, i.e. portions, wherein, if the catheter comprises several bendable segments, they may operate independently from each other. In this case for each of the segments the bending radii or bending angles may be extracted from the OSS data and fed into the one or several controlling units that are controlling the individual segments.

Moreover, a same segment of the interventional instrument may comprise several bending elements, in particular, several SMA wires, in order to enable bending in multiple directions. For instance, the control system can be extended to two or three actuators, i.e. bending elements, per segment for bending in one plane in two directions (two actuators) and bending in various planes (three actuators). To this end, radii of curvature or bending angles and azimuth angles (0 or 180° in case of two actuators, 0 to 360° in case of three actuators) are preferentially extracted from the shape sensor data.

Although in an above described embodiment an OSS fiber is centrally located within a catheter, the OSS fiber can also be located out of the center of the catheter.

Although in above described embodiments the interventional instrument is a catheter, in other embodiments the interventional instrument can also be another instrument like a guidewire. Moreover, although in above described embodiments a catheter steering is provided for vascular applications, the steering of the interventional instrument can also be provided for other applications like navigating interventional instruments in more delicate anatomical structures such as brains, lungs, et cetera. The interventional system and method preferentially use an interventional instrument having a long and slender body with bendable portions, wherein the shape of these bendable portions is controllable.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the determination of the degree of bending, the control of the bending element, et cetera performed by one or several units or devices can be performed by any other number of units or devices. These procedures and/or the control of the interventional system in accordance with the interventional method, in particular, the control of one or several bending elements of the interventional instrument based on OSS signals, can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to an interventional system for performing an interventional procedure. An interventional instrument like a catheter comprises a bendable portion, which is bendable by a bending element, and an OSS fiber for generating OSS signals being indicative of the degree of bending of the bendable portion. The actual degree of bending of the bendable portion is determined based on the generated OSS signals and the bending element is controlled depending on the actual determined degree of bending. By using OSS, the actual real degree of bending of the bendable portion of the interventional instrument can very accurately be determined. Moreover, since the bending element is controlled based on this very accurately determined degree of bending, the control of the bending element and, thus, of the interventional instrument can be very accurately.

1. An interventional system for performing an interventional procedure, the interventional system comprising:
a handheld interventional instrument comprising a bendable portion, wherein the interventional instrument is equipped with a bending element being adapted to bend the bendable portion and with an optical shape sensing fiber for generating optical shape sensing signals being indicative of the degree of bending of the bendable portion, wherein the bending element comprises a smart material connected to the bendable portion, wherein the smart material is adapted to change its spatial configuration depending on an external stimulus, in order to change the degree of bending of the bendable portion,
a bending determination unit for determining the degree of bending of the bendable portion based on the generated optical shape sensing signals,
a desired bending providing unit for providing a desired degree of bending,
a control unit for controlling the bending element in a control loop depending on the determined degree of bending and on the desired degree of bending such that the determined degree of bending is similar to the desired degree of bending by providing an external stimulus to the smart material.

2. The interventional system as defined in claim 1, wherein the interventional instrument is a catheter.

3. The interventional system as defined in claim 1, wherein the interventional instrument comprises several bendable portions and several bending elements for bending the several bendable portions, wherein the bending determination unit is adapted to determine the degrees of bending of the several bendable portions and wherein the control unit is adapted to control the respective bending element depending on the respective determined degree of bending.

4. The interventional system as defined in claim 1, wherein the interventional instrument comprises several bending elements for bending the same bendable portion of the interventional instrument, wherein different bending elements are adapted to bend the same bendable portion in different directions and wherein the control unit is adapted to control the bending elements depending on the determined degree of bending.

5. The interventional system as defined in claim 1, wherein the smart material includes a shape memory alloy and/or an electroactive polymer.

6. The interventional system as defined in claim 1, wherein the desired bending providing unit is adapted to provide a user interface for allowing a user to input the desired degree of bending and to provide the input desired degree of bending.

7. The interventional system as defined in claim 6, wherein the user interface is integrated in a handle of the interventional instrument.

8. The interventional system as defined in claim 1, wherein the interventional system further comprises a desired position providing unit for providing a desired position of the interventional instrument, wherein the desired bending providing unit is adapted to determine the desired degree of bending based on the provided desired position of the interventional instrument.

9. The interventional system as defined in claim 8, wherein the interventional system further comprises a position determination unit for determining the position of the interventional instrument and wherein the desired bending providing unit is adapted to determine the desired degree of bending further based on the determined position of the interventional instrument.

10. The interventional system as defined in claim 1, wherein the bending determination unit is adapted to determine the curvature and/or the radius of curvature and/or the bending angle of the bendable portion as the degree of bending.

11. (canceled)

12. A computer program for performing an interventional procedure, the computer program comprising program code means for causing an interventional system as defined in claim 1 to carry out the steps of an interventional method, when the computer program is run on a computer controlling the interventional system, the interventional method comprising:
generating optical shape sensing signals being indicative of a degree of bending of a bendable portion of a handheld interventional instrument which is equipped with an optical shape sensing fiber for generating the optical shape sensing signals and with a bending element for bending the bendable portion, wherein the bending element comprises a smart material connected to the bendable portion, wherein the smart material is adapted to change its spatial configuration depending on an external stimulus, in order to change the degree of bending of the bendable portion,
determining the degree of bending of the bendable portion based on the generated optical shape sensing signals by a bending determination unit,
providing a desired degree of bending by a desired bending providing unit, and
controlling the bending element in a control loop depending on the determined degree of bending and so the desired degree of bending such that the determined degree of bending is similar to the desired degree of bending by a control unit providing an external stimulus to the smart material.
